(19) United States
(12) Patent Application Publication
(10) Pub. No.: US 2015/0050331 A1
(43) Pub. Date: Feb. 19, 2015

(54) METHOD FOR MAKING A TOPICAL COMPOSITION COMPRISING GROWTH FACTORS DERIVED FROM HUMAN UMBILICAL CORD BLOOD PLATELETS

(71) Applicant: NOVO SOLUTIONS MD, LLC, PARADISE VALLEY, AZ (US)

(72) Inventor: ALVIN DAVID NEEDLEMAN, LAS VEGAS, NV (US)

(73) Assignee: NOVO SOLUTIONS MD, LLC, PARADISE VALLEY, AZ (US)

(21) Appl. No.: 14/527,284

(22) Filed: Oct. 29, 2014

Related U.S. Application Data
(63) Continuation-in-part of application No. 14/260,945, filed on Apr. 24, 2014.
Application No. 14/260,945 is a continuation-in-part of application No. 14/294,570, filed on Jun. 3, 2014.
Application No. 14/294,570 is a continuation-in-part of application No. 14/100,978, filed on Dec. 9, 2013.
(60) Provisional application No. 61/816,135, filed on Apr. 25, 2013.
(60) Provisional application No. 61/830,169, filed on Jun. 3, 2013.

(51) Int. Cl. A61K 38/18; A61K 9/00; A61K 9/127; C12N 5/078

(52) U.S. Cl. CPC A61K 38/1858; C12N 5/0644; A61K 38/18; A61K 9/0014; A61K 9/127; A61K 9/1277; C12N 2509/00
USPC 424/450; 435/173.7; 435/325; 514/8.2; 514/8.1; 514/8.9; 514/7.6

(57) ABSTRACT

The present invention is directed to a method of making a stable composition comprising growth factors, which may include platelet derived growth factors and transforming growth factors. The method comprises providing human umbilical cord blood plasma containing platelets, but substantially free of whole blood cells, and lysing the platelets to extrude growth factors into the plasma. The plasma may be obtained from multiple donors and pooled to form a homologous plasma mixture. In another embodiment, the growth factors are encapsulated in a liposome formed by a lipid bilayer, wherein the resulting composition remains stable and viable for at least 30 months. The present invention is also directed to the composition produced by the methods of the present invention and the process of applying the composition to a skin defect or wound.

### CROSS-REFERENCE TO RELATED APPLICATIONS

This application claims priority to and is a Continuation-in-Part of U.S. patent application Ser. No. 14/260,945, filed on Apr. 24, 2014, which claims priority to U.S. Provisional Patent Application Ser. No. 61/816,135, filed on Apr. 25, 2013; this application further claims priority to and is a Continuation-in-Part of U.S. patent application Ser. No. 14/294,570, filed on Jun. 3, 2014, which claims priority to U.S. Provisional Patent Application Ser. No. 61/830,169, filed on Jun. 3, 2013; this application further claims priority to and is a Continuation-in-Part of U.S. patent application Ser. No. 14/100,978, filed on Dec. 8, 2013, each of which is incorporated herein by reference in its entirety.

### STATEMENT REGARDING FEDERALLY SPONSORED RESEARCH OR DEVELOPMENT

Not applicable.

### BACKGROUND OF THE INVENTION

1. Field of the Invention

The present invention is related to the field of topical skin care and repair compositions.

2. Description of Related Art

The gradual development of facial wrinkles, whether fine surface lines or deeper creases and folds, is an early sign of accumulated skin damage and skin aging, which may be intrinsic and/or caused or accelerated by external factors. Other non-wound skin defects can also be intrinsic and/or caused or accelerated by external factors. For example, premature aging and wrinkling of the skin may be accelerated by excessive exposure to the sun and other damaging elements, overactive facial expression muscles, frequent use of tobacco products, poor nutrition, or skin disorders. Fine surface wrinkles that progress to deeper creases, deepening facial expression due to repeated skin folding, and deep folds which develop with one's maturity are visible changes which may combine to portray a less desirable appearance.

Various attempts at anti-aging skin care compositions have used botanicals, antioxidants, and biopeptides, among other things. Several invasive techniques are available in which substances are injected or implanted in the area of the skin which either temporarily weaken the muscles or act as skin volume fillers. However, invasive techniques are often risky and require the supervision or assistance of a physician, which can be inconvenient and costly, and non-invasive treatments have historically met with only minimal success. Regardless of the cause of facial creases or folds, safe and effective treatments for reduction or elimination of these problems have been exceedingly difficult to achieve. Thus, there remains a need for new and improved topical skin care compositions that are useful as an anti-aging composition.

There are also many existing techniques to address wound care and repair. The repair of wound tissue is an extremely complex biological process that can lead to the production of scar tissue. Thus, it has been a challenge to produce topical skin care compositions that are useful in treating and repairing wounds and reducing the appearance and the formation of scar tissue.

### BRIEF SUMMARY OF THE INVENTION

The present invention is directed to a method of making a stable composition comprising growth factors. In one aspect of the invention, the method includes the steps of providing human umbilical cord blood plasma containing platelets, wherein the platelet-containing plasma is substantially free of whole blood cells, lysing the platelets to extrude growth factors into the plasma, and encapsulating the growth factors contained within the plasma in a lipid bi-layer, wherein the resulting composition comprises encapsulated growth factors and remains stable and viable for at least 30 months.

In one aspect of the invention, the encapsulating step comprises adding propanediol and lecithin to the plasma to create one or more lipid bi-layers which form liposomes around the growth factors.

In another aspect of the invention, the human umbilical cord blood plasma is obtained from at least two or more donors.

One aspect of the invention is directed to a method for extruding growth factors from platelets contained in the cord blood plasma. In one such embodiment, the extruded growth factors are selected from the group consisting of platelet-derived growth factors and transforming growth factors.

In one aspect of the invention, the lysing step is selected from the group consisting of traumatic lysing and protein induction. In one embodiment of the invention where lysing occurs through traumatic lysing, the traumatic lysing may be selected from the group consisting of lysing by temperature shock, lysing by pH shock, lysing using high molecular weight fatty alcohols, and lysing by light emission.

In one embodiment of the invention where the traumatic lysing is lysing by temperature shock, the lysing method may include freezing the platelet-containing plasma to between −20° C. and −30° C., and rapidly thawing the frozen plasma to between 32° C. and 39° C.

In one embodiment of the invention where the traumatic lysing is lysing by pH shock, the method may include reducing the pH of the platelet-containing plasma to between 5.0 and 5.5. In one embodiment of the invention where the traumatic lysing is lysing by light emission, the method may include exposing the platelet-containing plasma to light emission in a visible range. In one embodiment of the invention where the lysing is by protein induction, the method may include the addition of thromboglobulins.

One aspect of the invention is directed to a method including the step of adding at least one matrikine peptide to the plasma. The matrikine peptide may be selected from the group consisting of gene receptor-stimulating matrikine peptides and matrix-derived peptides that regulate cell activity. In one embodiment, the matrikine peptide may be selected from the group consisting of Pal-GHK, Pal-GQPR. In one such embodiment, the matrikine peptide is in a solution comprising 0.25% to 4.0% matrikine peptides (v/v) and the matrikine peptide solution comprises between 0.01% and 0.09% (v/v) of the platelet-containing plasma composition.

One aspect of the invention is directed to the composition produced by a method of the present invention. In one such embodiment, the composition may include one or more additives selected from the group consisting of preservatives, emollients, delivery enhancers, penetrability enhancers, and moisturizers.

One aspect of the invention is directed to a method for applying a composition produced by a method of the present invention to a skin defect or wound on a subject's skin. In one such embodiment, the skin defect or wound is selected from the group consisting of a cosmetic skin defect, a traumatic skin defect, a chronic defect, a non-healing ulcer, an acute wound, a wound resulting from non-surgical/accidental trauma, a wound resulting from surgical trauma, a wound resulting from a chronic disease state, a wound resulting from topical irritation, trauma depleted fibroblasts, trauma depleted collagen, trauma depleted elastin, depleted adhesive plaque at the dermal/epidermal junction, damage caused by age-related skin deterioration, collagen alignment, scarring, scar formation, stretch marks, keloids, non-healing cratered ulcers, diabetic ulcers, cratered ulcers, diabetic neuropathies, hardened-cracked skin, hardened cracked heel tissue, and pressure ulcers, fine lines, wrinkles, and skin sagging.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 depicts a the overall skin condition of the upper surface of the foot depicted in FIG. 2.

FIG. 2 depicts a wound prior to application of a composition of the invention.

FIG. 3 depicts the wound of FIG. 3 application of a composition of the invention.

FIG. 4 depicts a wound prior to application of a composition of the invention.

FIG. 5 depicts the wound of FIG. 4 after 3 weeks of applying a composition of the present invention.

FIG. 6 depicts the wound of FIG. 4 after 12 weeks of applying a composition of the present invention.

FIG. 7 depicts a wound prior to and after application of a composition of the present invention.

### DETAILED DESCRIPTION OF PREFERRED EMBODIMENT

The present invention is directed to a method of making a topical composition comprising growth factors. In one embodiment of the present invention, the method comprises the steps of providing human umbilical cord blood plasma containing platelets, but substantially free of whole blood cells, and lysing the platelets to extrude growth factors into the plasma. The growth factors are preferably selected from platelet derived growth factors and transforming growth factors. The growth factors are preferably selected from the group consisting of FIGF, PDGFA, PDGFB, PDGFC, PDGFD, PGF, VEGF, VEGF41, VEGFB, or VEGFC. In one embodiment of the invention the growth factors are encapsulated in a lipid bilayer and the resulting composition remains stable and viable for at least 30 months. The present invention is also directed to the composition produced by the methods of the present invention. In one embodiment, the composition is designed for topical application to a skin defect or wound on a subject's skin, and the present invention is also directed to the process of applying the composition to a skin defect or wound.

Methods for obtaining human umbilical cord plasma are known in the art. In one embodiment of the invention, the human umbilical cord blood is collected with a needle from the umbilical vein into a cord blood collection bag. The cord blood collection bag preferably contains an anti-coagulant such as citrate phosphate dextrose. The amount of anti-coagulant can be calculated by known methods in the art. In one embodiment, 35 mL of citrate phosphate dextrose is added to a 250 mL cord blood collection bag which generally allows the collection of 210 mL of cord blood. Generally a 75 mL sample is collected.

The blood cord bag is designed for safe use within the sterile surgical field. Suitable human umbilical cord blood collection bag sets are commercially available and known in the art. A unique collection bag shape maximizes the recovery of cord blood. In one embodiment, the collection bag preferably contains an in-line sterile air vent that permits the recovery of blood without the need to strip tubing, a process that can lead to cell damage. A tethered cap on the vent can ensure that there are no loose parts in the sterile field. The bag also preferably includes in-line spike entry port/tubing compatible with sterile connection devices. This permits connection to other blood processing components by any means. It also provides a back-up system for sterile connection device failure or lack of availability. The bag preferably includes a multiple-use sampling port to permit pulling samples for testing and facilitate the addition of solutions such as sedimenting agents.

The needle preferably contains a finger-friendly contoured hub with a “bevel up” indicator to facilitate a secure needle stick into the umbilical vein. A needle guard, such as that sold by Donor Care, is a simple but effective method for protecting against accidental needle-stick injuries to collection staff.

A sedimentation agent may be added to the collected cord blood. The sedimentation agent reduces the volume of cord blood by enhancing sedimentation of red blood cells by Rouleaux formulation. The sedimentation agent may be selected from the group consisting of hydroxyethyl starch (such as that sold under the trade name Hespan by B. Braun Medical, Inc.), succinylated gelatin, pentastarch and the proprietary solution contained in the PrepaCyte CB system sold by BioE LLC. When hydroxyethyl starch is used, the amount to be added may be calculated by dividing the volume of cord blood in the collected sample by five to obtain the amount of hydroxyethyl starch to be added. Preferably the sedimentation agent is injected into the blood collection bag prior to centrifugation.

The cord blood may be centrifuged to separate the cord blood plasma into three components, namely a plasma layer, a buffy coat layer, and a cellular pellet of red blood cells. In one embodiment, the cord blood is centrifuged in a bag in a Sorvall RC-3B centrifuge at a speed between 1500 and 2500×G, with the value 6.673×10⁻¹¹ NM² KG⁻², for 9 to 15 minutes at a temperature of 6° C. to 15° C. In one such embodiment, the plasma is centrifuged at 2000×G for 12 minutes at 8° C. to 12° C. The three components may be separated into three bags during the centrifugation step. The separated components may be allowed to settle, preferably for 35 minutes, but no longer than 2 hours after centrifugation. The plasma layer and supernatant in the buffy coat layer are collected and combined. Preferably the collecting step utilizes aspiration, in which the residual cellular pellet containing whole cellular materials in the buffy coat bag is aspirated and discarded.

The combined plasma layer and buffy coat supernatant layer are combined and may be centrifuged a second time, preferably at similar conditions, to remove remaining cellular components. In one embodiment, the second centrifugation may be performed in a Sorvall RC-3Cplus centrifuge at 2,000×G for twelve minutes at 8° C. to 12° C. The supernatant is again collected. The approximate platelet content in the collected platelet-containing plasma is approximately 6,000 platelets per mL. The anti-coagulant is substantially removed with the whole cellular materials in the red blood cell layer and cellular pellet of the buffy coat layer. As a result, the platelet-containing plasma is substantially free of whole blood cells and anti-coagulant. Because there are no longer red blood cells in the plasma, there is no need to add anti-coagulants in later steps of the production process. This process allows processing of low volume cord blood samples, samples with low total nucleated cell count, and samples received between 48 and 72 hours after collection.

In one embodiment, the platelets are concentrated in the plasma by repeated centrifugation. In one such embodiment, the platelet-containing plasma may be centrifuged an additional 3 to 5 times at 1500-3500×G for 5-15 minutes at a temperature of 6° C. to 30° C. The upper portion of the plasma is aspirated and discarded. The lower portion (approximately 60%) is recentrifuged and reaspirated. The centrifugation/aspiration process is repeated until the final desired concentration is reached, preferably greater than 200,000 platelets/mL.

In one embodiment of the present invention, the platelet-containing plasma is frozen, preferably at −20° C., to preserve the platelet-containing plasma for further processing and thawing prior to use. Samples from individual donors are saved in separate sample units pending testing for disease markers.

In one embodiment of the present invention, a portion of the cord blood is removed from each collected sample for standardized testing for the presence or absence of disease markers. Such disease markers may include Hepatitis C Aby, Hepatitis core Aby, RPR, Syphilis screen, HIV ½ plus 0 Aby, HTLV I/II Aby, CMV IgM Aby, Hepatitis C Ag by NAT, Hepatitis B surface Ag, T. Cruzi (Chagas) Aby, HIV Ag by NAT, West Nile Virus by NAT, bacterial contamination, and fungal contamination. The samples may also be tested for pH, specific gravity and viscosity.

In one preferred embodiment, platelet-containing plasma from multiple donors is combined prior to further processing. Platelet-containing plasma from at least 2 donors may be combined, and in certain embodiments samples from at least 15 donors are combined. In the embodiment wherein the platelet-containing plasma was frozen, the plasma is collected and thawed to allow combination and mixing. This can be done by thawing the plasma in a 37° C. water bath for 3 to 5 minutes. The collected and thawed units are combined. In certain embodiments this may be done in a 1 liter Nalgene bottle. The combined units are mixed well to produce a homologous product.

The platelet-containing plasma may be tested for microbial contamination by methods known in the art, such as by use of the BacT/Alert media sold by bioMéricux, Inc.

One or more matrikine peptides may be added to the platelet-containing plasma, preferably after the plasma from multiple donors has been combined. The matrikine peptides are preferably gene receptor-stimulating matrikine peptides and/or matrix-derived peptides that regulate cell activity. Matrikine peptides refer to oligopeptides and protein fragments that are generated during the macromolecule breakdown process. Matrikine peptides can result from partial proteolytic cleavage of constituents of the extra cellular matrix. Some of the resulting fragments possess stimulating and signaling activity in a feedback loop, which can initiate the repair process of the tissue matrix. It is believed these peptide fragments participate in wound healing as natural, non-toxic, locally acting, highly potent messengers, wherein the matrikine peptides act in synergy to restore and maintain the skin's youthful appearance. One mechanism of action is thought to be activation of the neosynthesis of extracellular matrix molecules providing visible anti-wrinkle efficacy. Further, as messenger molecules, the matrikine peptides are capable of regulating cell activities. They can interact with specific receptors to activate certain genes involved in extracellular matrix renewal and cell proliferation. Matrikine peptides have been shown to elicit chemotaxis, promote cell cycle progression, induce release of proteolytic enzymes and cause the production of cytokine and growth factors. For example, pal-GQPR and pal-GHK are believed to activate the genes involved in de novo matrix synthesis (particularly collagen and fibronectin) and thereby promote proliferation of keratinocytes and fibroblasts and proteoglycan anchoring, and ultimately promote repair of damaged matrix. The matrikine peptides are preferably selected from the group consisting of Pal-GHK (palmitoyl oligopeptide) and Pal-GQPR (palmitoyl tetrapeptide-7. The capital letters indicate the IUPAC one-letter code for the amino acids comprising the peptides. In one embodiment of the invention, at least two matrikine peptides are added.

In certain embodiments, the matrikine peptides will be in a solution comprising 0.2% to 4.0% matrikine peptides (v/v) prior to addition into the platelet-containing plasma. The matrikine peptide solution may contain glycerin, water, butylene glycol, carbomer and polysorbate-20. After the matrikine peptide solution is added to the platelet-containing plasma, the matrikine peptide solution preferably comprises between 0.1% and 0.9% (v/v) preferably 0.5% of the platelet-containing plasma composition. In one preferred embodiment, 1 mL to 5 mL of the matrikine peptide solution is added to 1 L of the platelet-containing plasma. The platelet-containing plasma containing the matrikine peptides may be separated into aliquots, preferably 50 mL, frozen at −20° C. and stored. Plasma may be transferred to a −80° freezer for transportation and long-term storage.

At least a portion of the platelets in the platelet-containing plasma are lysed to extrude growth factors into the plasma. The extruded growth factors are preferably selected from the group consisting of platelet derived growth factors and transforming growth factors. In one embodiment, the growth factors are selected from the group consisting of FIGF, PDGFA, PDGFB, PDGFC, PDGFD, PGF, VEGF, VEGF41, VEGFB, and VEGFC. Exemplary growth factors include TGF-B, VEGF, HGF, KGF, Il-6, Il-7, Il-8, bFGF, IGF1, PDGF, TGF-B2, TGF-B3, and granulocyte-monocyte colony-stimulating factors. PDGF growth factors will generally be present in one of three dimeric forms, PDGF-AA, PDGF-BB and PDGF-AB. PDGF-AA is one of the preferred PDGFs to be extruded.

In certain preferred embodiments of the invention, the lysing step may be performed by traumatic lysing and/or protein induction. More than one traumatic lysing method may be used and/or traumatic lysing may be combined with protein induction. Traumatic lysing is a lysing method that causes the platelet walls to rupture by introducing trauma to the platelets. Traumatic lysing processes may be selected from the group consisting of lysing by temperature shock, lysing by pH shock, lysing using high molecular weight fatty alcohols and lysing by light emission.

Lysing by temperature shock is preferably performed by freezing the platelet-containing plasma to between −20° C. and −30° C., or using the previously frozen plasma, and rapidly thawing the frozen plasma between to 32° C. and 40° C. The rapid thawing preferably occurs within 30 to 40 minutes. Thawing may be conducted in a water bath. Alternatively, the frozen plasma may be added directly to a liquid formulation having an original temperature of 30° C. to 50° C. Such liquid formulation preferably comprises water and/or other components that will be contained in the final composition.

In the embodiment in which traumatic lysing is lysing by pH shock, the pH of the platelet-containing plasma is reduced to between 5.0 and 5.5. This may be achieved by adding the platelet-containing plasma into a liquid formulation having a pH between 5.0 and 5.5. Frozen samples are thawed before adding to the liquid formulation. Such liquid formulation preferably comprises water and/or other components that will be contained in the final composition. The pH shock is preferably performed at temperatures between 25° C. to 90° C.

In embodiments wherein traumatic lysing is achieved using high molecular weight fatty alcohols, the platelet-containing plasma is exposed to anionic, cationic or non-ionic high molecular weight fatty alcohols, preferably at a temperature between 70° C. and 95° C., more preferably between 70° C. and 80° C. Suitable high molecular weight fatty alcohols include polyclyceryl-3, methylglucose distearate, distearyldimonium chloride, and cetearyl palmitamidopropyltrimonim chloride. The fatty alcohols also serve as emulsifying agents. The platelet-containing plasma may be exposed to a solid high molecular weight fatty alcohol by combining the platelet-containing plasma, fatty alcohol, preferably in a liquid formulation. Frozen samples are thawed before adding the fatty alcohol. Preferably the platelet-containing plasma comprises 0.1% to 10% (v/v) of the combination and high molecular weight fatty alcohols comprise 3% to 10% (v/v) of the combination. The remainder of the composition preferably comprises water and/or other components that will be contained in the final composition. The composition is preferably subjected to emulsification, homogenization or high shear mixing.

In embodiments wherein traumatic lysing is lysing by light emission, the platelet-containing plasma is exposed to infra-red laser emission in the visible range, preferably between 495 nm and 700 nm at 35 mW-50 mW. The platelet-containing plasma may be exposed for 3 to 9 minutes, preferably 5 minutes, while rotating, wherein the rotation speeds preferably range between 0.5 RPM to 8 RPM, preferably at 1 RPM.

In certain embodiments the lysing is achieved through protein induction rather than traumatic lysing. This method is particularly beneficial for use in preparing compositions that will be used in treating intact skin trauma, such as aging. Protein induction may be achieved by causing the extrusion of growth factors following the addition of a thromboglobulin to the platelet-containing plasma. Frozen samples are thawed before adding the thromboglobulins. Thromboglobulins are preferably added in an amount of 0.01% to 0.5% (v/v) of the platelet-containing plasma.

The growth factors extruded into the plasma are then encapsulated in a lipid bi-layer. The resulting composition remains stable and viable for at least 30-36 months. The lipid encapsulation protects against oxidation and negative interaction with ingredients in the composition, the environment, skin pH and polar changes. Preferably the growth factors are encapsulated in one or more lipid bi-layers that form liposomes around the growth factors. In certain embodiments, a phospholipid forms the bi-layer with deionized water, which is then added to the platelet-containing plasma and processed to form a liposome. The phospholipid is preferably selected from the group consisting of lecithin, phosphatidylcholine and phosphatidylserine.

In one embodiment, the plasma is combined with deionized water, propanediol and lecithin. The deionized water is preferably added to a total volume of 10-20% of the total composition. A propanediol and lecithin solution is preferably added in an amount approximately equal to the amount of the platelet containing plasma, preferably each comprise 40% to 45% of the total volume. The plasma comprises between 40% and 45% of the composition at the encapsulation step. In this process, the lipid bi-layer formed by the propanediol and lecithin is capable of producing a liposome in 10 to 20 minutes.

The encapsulation step may be performed by any method known in the art. In one embodiment, the encapsulation step is preferably performed by mixing the components in a four bladder mixer at 600-1500 rpm for 3 to 20 minutes to produce liposomes entrapping in the growth factors. The liposomes are preferably non-geometric and randomly shaped, comprising microscopic hollow soft vesicles composed of one or more phospholipid bi-layers surrounding an aqueous core. Thus, the liposomes may be unilamellar or multilamellar. The liposomes are able to entrap the hydro-soluble active ingredients in the internal cavity and lipid-soluble active ingredients in the lipid bi-layer(s) of the membrane. Because of their cell-like composition, liposomes are efficient biometric vectors highly adapted for cosmetic/topical products to improve ingredient percutaneous absorption and efficacy. They act as a non-skin-irritating penetration enhancer and present a higher ability to cross a cutaneous barrier and diffuse through skin layers, leading to the improvement of bioavailability.

The present invention is also directed to the composition produced by the methods of the present invention. The composition is preferably a topical formulation designed for topical application to a subject's skin. In one embodiment, the composition comprises a pharmaceutically, physiologically, cosmetically, and dermatologically acceptable vehicle, diluent or carrier. The composition preferably comprises 0.1%-10.0% plasma and 99.9%-90% vehicle and other ingredients.

The composition of the present invention may also include one or more additives selected from the group consisting of preservatives, emollients, delivery enhancers, penetrability enhancers and moisturizers. In certain embodiments, one or more additives can be combined without the growth factors to form a composition designed for topical application to a subject's skin.

Preservatives may be selected from the group consisting of antibacterials, and anti-oxidants, anti-fungals, anti-microbials and aromatic oils. Preservatives may include phenoxyethanol, sorbic acid, benzyl alcohol, dehydroacetic acid and their derivatives. In the embodiment wherein the preservative is phenoxyethanol and sorbic acid, the preservative is added in a range from 0.5% to 1.5% (v/v). In the embodiment where benzyl alcohol dehydroacetic acid (DHA) is added, the preservative is preferably added in a range of 0.1% to 0.9% (v/v).

Emollients, delivery enhances, and penetrability enhancers may be utilized in the composition. In one embodiment, chemical delivery enhancers are added to the composition of the present invention. In one such embodiment, the chemical delivery enhancers can be selected from the group consisting of isosorbides, preferably in a range of 0.1% to 10.0% v/v of the total composition, dimethyl isosorbides, preferably in a range of 0.1% to 10.0% v/v of the total composition, aloe vera concentrated extract, preferably in a range of 0.1% to 5.0% v/v of the total composition, polysorbate-20 (tween), preferably in a range of 0.1% to 10.0% v/v of the total composition, polysorbate-60, preferably in a range of 0.1% to 10.0% v/v of the total composition, hyaluronic acid, preferably in a range of 0.001% to 4.0% v/v of the total composition, and anionic polysaccharides, such as polyglucosamines, preferably in a range of 0.1% to 5.0% v/v of the total composition. One preferred polyglucosamine is poly-D-glucosamine. The composition comprising the chemical delivery enhancers is preferably adjusted to a pH of 5.7-6.0.

In one preferred embodiment, penetrability enhancers are non-ionic crystal forming emulsifiers. Suitable emulsifiers may be cationic, non-ionic or anionic and may include sorbitan stearate and sorbityl laurate, preferably in a range of 0.5% to 10.0% v/v of the total composition.

In another embodiment, penetrability is enhanced by the use of iontophoresis with negative and positive pads applied to the skin. In one such embodiment, the positive pad is charged with the composition of the present invention, and current is applied at a rate of 50 mA for a period ranging from 30 seconds to 4 minutes.

In yet another embodiment, penetrability is enhanced by ionization using a positively charged probe. The probe is preferably utilized at a frequency of 30 kHz+20% and an ionization output of 0.1 mA+20% for 2-3 minutes.

In another embodiment, penetrability is enhanced by sonic skin absorptions. In one such embodiment the sonic skin absorptions comprise micro-vibrations at 250 to 350 vibrations/second, preferably 300 vibrations/second.

The composition of the present invention may also comprise moisturizers. The moisturizers may be osmotic carbohydrate moisturizers. The moisturizers may be selected from the group consisting of L-fructose, D-galactose, galacturonic acid, and erythritol-homarine HCl.

Other additives may include one or more of botanicals, including centella asiatica, goji berry extract, rosemarinus officinalis, aloe vera; amino acids, including 1-proline, 1-arginine; essential oils, including jojoba oil, macadamia nut oil, almond oil, squalane, evening primrose, hempseed oil, borage oil; ceramides, including ceramide 3-ceramide 6 II-ceramide 1-phytosphingosine-cholesterol-sodium lauroyl lactylate; vitamins, including niacinamide and magnesium acorbyl phosphate; anti-infectives, antiseptics and anti-fungals including melalueca alternifolia; anti-dermatophytics, including terpenoids, including terpinen-4-ol; matrix formers; epithelialization formers; barrier cross polymers, including cyclopentasiloxane and dimethicone/vinyl dimethicone cross-polymers; high molecular weight alcohols, including behentrimonium; methosulfate-cetyl alcohol-butylene glycols; inhibitors of trypsin and chymotrypsin, including Phaseolus lunatis and rutin and other proteins and antioxidants; peptides including N-acetyl tyrosyl arginyl hexadecyl ester; neuropeptides including acetyl hexapeptides; liposomes and lipids including propanediols and lecithins; and myristamidopropyl PG-dimonium chloride phosphate.

With respect to neuropeptides, such as the acetyl hexapeptides, the transmission of nerve signals across cholinergic synapses involves the presynaptic release (or secretion) of acetylcholine (ACH) which, by acting on specific sites of the postsynaptic membrane, induces changes in the postsynaptic transmembrane potential. According to the vesicle hypothesis, ACH is packed inside synaptic vesicles which release their contents from the nerve terminal on contact with the presynaptic membrane. The process is blocked by the enzyme acetylcholinestrase causing a blockage of afferent signals (i.e., neuropathic pain). Neuropeptides, specifically acetyl hexapeptides, can be responsible for an increase in the acetylcholinestrase causing inhibition of acetylcholine resulting in afferent signal blockage and blockage of neuropathic pain.

The dry green bean extract, or Phaseolus lunatus, contains a small cysteine protein with a molecular weight of 8,000 Daltons which inhibits tryptase and chymase. This small, cysteine-rich protein is stabilized by disulfide bridges which are important in terms of stability and efficacy. Its three-dimensional structure has 2 trypsin and chymotrypsin recognition sites, thus conferring an inhibitory effect on the protein. The protein is a member of the Bowman-Birk protease inhibitor (BBI) class, the leading protein of which is the soybean Bowman-Birk protease inhibitor. In the skin, collagenase activity depends on the conversion of procollagenase into active collagenase. Conversion is dependent on trypsin. Inhibiting trypsin thus limits collagenase activity. It is also known that MMP2 and MMP9 are activated by trypsin. Chymotrypsin, normally present in the skin and abundantly released by mast cells, is to be markedly reduced during cell cicatrization in order to promote de novo synthesis of the matrix. Under those conditions, a dual trypsin-chymotrypsin inhibitor enables restoration of tissue homeostasis.

Rutin possesses antioxidant properties of value in inflammatory stress and an ability to stabilize mast cells as shown by third party studies showing that in the presence of rutin, activated mast cells no longer release histamine and the surface antigen presentation which reflects mast cell activation is antagonized by rutin. Moreover, third parties have identified a new property of rutin: inhibition of leukocytic elastase. This strengthens the protective properties with respect to the extracellular matrix.

The present invention is also further directed to a method for applying a composition of the present invention to a subject in need thereof by applying the composition to a skin defect, a wound on a subject's skin or to intact skin to prevent defects or wounds. The composition is preferably applied more than once. The composition is applied in an amount effective for improving or preventing the wound or defect. It is believed that the composition of the present invention will penetrate to the extra cellular matrix, basement membrane or dermal epidermal junction of the skin.

The skin defect or wound may be a cosmetic skin defect, a traumatic skin defect, a chronic defect, a non-healing ulcer, an acute wound, a wound resulting from non-surgical/accidental trauma, a wound resulting from surgical trauma, a wound resulting from a chronic disease state, a wound resulting from topical irritation, trauma depleted fibroblasts, trauma depleted collagen, trauma depleted elastin, depleted adhesive plaque at the dermal/epidermal junction, damage caused by age-related skin deterioration, collagen alignment, scarring, scar formation, stretch marks, keloids, non-healing cratered ulcers, diabetic ulcers, cratered ulcers, diabetic neuropathies, hardened-cracked skin, hardened cracked heel tissue, and pressure ulcers, fine lines, wrinkles, or skin sagging. When the skin defect results in intact skin, the composition preferably includes thromboglobulins.

Application of the composition can provide various positive effects on skin defects and wounds, and in some cases can repair wounds and defects.

Application of the composition of the present invention may have a soothing affect and reduce pain. This may be achieved by stimulating genetic receptors, enhancing regulation of cellular made messages via the membrane receptors of keratinocytes, enhancing regulation of epigenetic mechanisms, stimulating sirtuins-1 and inhibiting protoglandin.

Application of the composition of the present invention may also result in the prevention of scar formation. This may be achieved by modulating stress at the wound site periphery by introducing an inter-tissue osmotic flow, interfering with the scar forming proteolytic enzymes (such as tryptase, chymase) or stimulating the inhibition of leucocytic elastatse blocking mast cell activity (such as may be achieved using rutin).

The application step can also enhance the functional capacity of skin layers, enhance secretion of growth factors in skin layers, enhance the attraction of macrophages and improve circulation to enhance skin regeneration, viability and elasticity. The composition of the present invention can also improve moisture uptake and retention in the skin by protecting against hypertonic and hypotonic stress, deccication and dehydration stress, enhancing aqua homeostasis of the skin cells, providing a barrier allowing the elective passage of gasses while inhibiting tissue and moisture loss, progressively hydrating the different layers of the skin, softening hard tissue, and adding water-binding cells to the extracellular matrix.

The application step may enhance repair and reconstruction of the extracellular matrix and normal skin architecture, mitogenic activity, procollagen production, collagen production, reactivation of trauma fatigued fiberblasts, reorganization of fibrils, and increased glycosaminoglycans as a result of growth factor stimulation of progenitor cells and stem cells. The application step may also stimulate genetically expressed Mesenchymal stem cell activity to stimulate extra cellular matrix construction and increased local vascularization (angiogenesis) and increased cell reproduction.

Mesenchymal stem cells (“MSC's”) are multi-potent (pluripotent) cells which can be obtained from several adult and fetal tissues including human umbilical cord units. Umbilical cord tissue (UC) is richer in MSC's than umbilical cord blood (UCB). Third party studies have demonstrated that a comparison of the exonic protein-coding (spliced) and intronic (non-spliced) noncoding RNA (ncRNA) expression profiles of MSC's from match-paired UC and UCB samples, harvested from the same donors, processed simultaneously and under the same culture conditions. The patterns of intronic ncRNA expression in MSC's from UC and UCB paired units were highly similar, indicative of their common donor origin. The respective exonic protein-coding transcript expression profiles, however, were significantly different. Hierarchical clustering based on protein-coding expression similarities grouped MSC's according to their tissue location rather than original donor. Genes related to systems development, osteogenesis and immune system were expressed at higher levels in UCB, whereas genes related to cell adhesion, morphogenesis, secretion, angiogenesis and neurogenesis were more expressed in UC cells. These molecular differences verified in tissue-specific MSC gene expression may reflect functional activities influenced by distinct niches and should be considered important when developing formulations involving growth factor stimulated MSC's from combined (homologous) donor sources.

Although not wanting to be bound by a single theory, it is believed application of the composition of the present invention may result in the activation of platelet surface receptors in broken or abraded skin of the subject, causing the trauma-stimulated release of a protein triad comprising thromboglobulins. Third party studies have demonstrated that platelet responses to thrombin are at least partly mediated by a G-protein-coupled receptor whose NH₂ terminus is a substrate for thrombin. The location of thrombin receptors in resting platelets are redistributed during platelet activation revealing several new aspects of thrombin receptor biology. On resting platelets, approximately two-thirds of the receptors are located in the plasma membrane. The remainder are present in the membranes of the surface connecting system. However, when platelets are activated by ADP or a thromboxane analog, thrombin receptors that were initially on the surface connecting system are exposed on the platelet surface, increasing the number of detectable receptors by 40% and presumably making them available for subsequent activation by thrombin. Platelet activation by thrombin rapidly abolishes the binding of the antibodies whose epitopes are sensitive to receptor cleavage and leaves the platelets in a state refractory to both thrombin and the antagonist peptide, SFLLRN. This can be accomplished by a 60% decrease in the binding of receptor antibodies directed COOH-terminal to the cleavage site, irrespective of whether the receptors are activated proteolytically by thrombin or nonproteolytically by SFLLRN. The loss of antibody binding sites cause by thrombin is due in part to receptor internalization and in part to the shutting of thrombin receptors into membrane microparticles, especially under conditions in which aggregation is allowed to occur. However, at least 40% of the cleaved receptors remain on the platelet service. Lacking the ability to synthesize new receptors, and lacking cellular reserve of preformed receptors comparable to that found in indothelial cells, platelets are unable to repopulate their surface with intact receptors following exposure to thrombin. This difference underlies the ability of indothelial cells to recover responsiveness to thrombin rapidly while platelets do not, despite the presence on both of the same receptor for thrombin.

The platelet-derived growth factor (PDGF) family consists of PDGF-A, -B, -C and -D, which form either homo- or heterodimers (PDGF-AA, -AB, -BB, -CC, -DD). The four PDGFs are inactive in their monomeric forms. PDGF subunits -A and -B are important factors regulating cell proliferation, cellular differentiation, cell growth. The PDGFs bind to the cellular surface protein tyrosine kinase receptors (PDGFR). There are two forms of PDGFR receptor-α and -β, each encoded by a different gene. These two receptor isoforms dimerize upon binding the PDGF dimer, leading to three possible receptor combinations, namely -αα, -ββ and -αβ. The extracellular region of the receptor consists of five immunoglobulin-like domains while the intracellular part is a tyrosine kinase domain. The ligand-binding sites of the receptors are located to the three first immunoglobulin-like domains. PDGF-CC specifically interacts with PDGFR-αα and -αβ, but not with -ββ, and thereby resembles PDGF-AB. PDGF-DD binds to PDGFR-ββ with high affinity, and to PDGFR-αβ to a markedly lower extent and is therefore regarded as PDGFR-ββ specific. PDGF-AA binds only to PDGFR-αα. PDGF-BB is the only PDGF that can bind all three receptor combinations with high affinity.

Dimerization is a prerequisite for the activation of the kinase. Kinase activation is visualized as tyrosine phosphorylation of the receptor molecules, which occurs between the dimerized receptor molecules (transphosphorylation). In conjunction with dimerization and kinase activation, the receptor molecules undergo conformational changes, which allow a basal kinase activity to phosphorylate a critical tyrosine residue, thereby “unlocking” the kinase, leading to full enzymatic activity directed toward other tyrosine residues in the receptor molecules as well as other substrates for the kinase. Expression of both receptors and each of the four PDGFs is under independent control, giving the PDGF/PDGFR system a high flexibility. Different cell types vary greatly in the ratio of PDGF isoforms and PDGFRs expressed. Different external stimuli such as inflammation, embryonic development or differentiation modulate cellular receptor expression allowing binding of some PDGFs but not others. Additionally, some cells display only one of the PDGFR isoforms while other cells express both isoforms, simultaneously or separately.

The following examples are directed to various exemplary embodiments of the compositions of the present invention and their use in accordance with the present invention.

### Example 1

Preparation of an exemplary composition of the present invention

A topical composition of the present invention may be formed by the following exemplary method, wherein trade names and/or manufacturer are listed in parentheses:

Cetearyl alcohol and behentrimonium methosulfate (Incroquat behenyl TMS 50 obtained from Croda Chemicals Europe) is added to deionized water, followed by cetearyl alcohol, polysorbate 60, and sorbitan stearate-sorbityl laurate, and the mixture is melted uniformly in a jacketed kettle to form to a homogenous phase A mixture at 80° C. The phase A mixture is held at 80° C. The kettle settings are wiper/scraper: 23.60, agitator: 27.9 and diffusion 4.32

Dimethyl isosorbide (Grant Industries), PPG-3 benzyl ether myristate (Croda), Cyclopentasiloxane-995 (BASF chemical division), triglyceride, phosphatidylcholine (Phonal 75-Lipo), evening primrose oil, borage oil, hemp seed oil, triethanolamine (TEA), D-panthenol, vitamin A microcaps (retinol cp-ch) and melaleuca alternifolia are added to a jacketed kettle at medium to medium-high speed at 50° C. to form a homogeneous phase B mixture. The phase B mixture is held at 50° C. under continued agitation at medium diffusion speed. The diffusion head is ⅓ diameter of the mixing vessel.

Sodium hyaluronate powder is combined with deionized water in a diffusion mixer at medium to medium-low speed to solubilize the sodium hyaluronante and form a homogeneous mixture at ambient temperature. Aloe vera 200× powder, Poly D-Glucosamine, L-proline, niacinamide, magnesium ascorbyl phosphate, lacto ceramide, centella asiatica, goji fruit extract and Bio-saccharide-gum (Solabia Group) are added sequentially to a lined plastic drum at medium to medium-low speed at ambient temperature to form a homogenous phase C mixture.

Phenoxyethanol SA, benzylacohol DHA, a mixture of butylene glycol, water, cetyl hydroxyetylcellulose, rutin, palmitoyl tripeptide-1, palmitoyl tetrapeptide-7 and Phaseolus lunatus (green Bean) 9 (Registril obtained from Sederma), Rosmarinus officinalis, a mixture of cyclopentasiloxane, dimethicone/vinyl dimethicone cross-polymer (Chemtec), and a mixture of glycerin, Water, butylene glycol, carbomer, polysorbate 20, palmitoyl tripeptide-1 and palmitoyl tetrapeptide-7 are mixed at temperatures under <30°-45 C to form phase D.

A plasma composition produced by the method of the present invention comprising growth factors derived from human umbilical cord platelets and matrikine peptides is combined with Pro-lip neo (propanediol and lecithin) obtained from Lucas Meyer cosmetics in a four bladder mixture at 500-1500 rpm for 5-25 minutes to form phase E liposomes entrapping the growth factors. The propanediol/lecithin formulation and plasma composition comprising the growth factors and matrikine peptides are provided in equal amounts to form 90% of the phase E composition and deionized water is added to comprise the remaining 10% of the phase E composition.

The jacketed kettle holding phase A is cooled to 50° C. and the phase B mixture is added at 3000-4000 rpm speed to form an emulsion. The temperature is lowered to ambient temperature and the phase C mixture is then added at 3000-4000 rpm to maintain the emulsion. The temperature is lowered below 30° C. and the phase D mixture is then added at 3000-4000 rpm speed to maintain the emulsion. The liposomes formed in phase E are added to the phase A, B, C, D mixture at 50° C.

The combined A, B, C, D, E emulsion is run through a Dynashear vertical homogenizer for one (1-3) passes depending on the final aesthetics and density desired, from a light to heavy topical cream. The homogenizer settings are pump pressure: 50; flow valve: half open; speed: 3000 RPM.

### Example 2

Treatment using an exemplary composition of the present invention

A composition of present invention was applied to chronic wounds that had failed to heal under standard protocols. The composition was applied at each dressing change, or up to 2-3 times daily, for up to 90 days.

The composition used in the study contained platelet derived growth factors and transforming growth factors extruded from platelets contained in human umbilical cord plasma. The platelets were lysed by temperature shock to extrude the growth factors as described herein. The growth factors were encapsulated within a lipid bi-layer formed by propanediol and lecithin in deionized water to form liposomes around the growth factors.

The encapsulated growth factors were added to a vehicle comprising the following ingredients: Cetearyl Alcohol, Polysorbate 60; Incroquat behenyl TMS-50; Butyrospermum Parkii; Glycerine; PPG-3 benzyl ether myristate; Cyclopentasiloxane; Squalane; Triethanolamine; Macrogolglycerol hydroxystearate, PEG-40; Triglyceride; a mixture of Glycerin (and) Butylene Glycol (and) Aqua (water) (and) carbomer (and) Polysorbate-20 (and) palmitoyl oligopeptide (and) palmitoyl Tetrapeptide-7; Hexapeptide-7; a mixture of Butylene Glycol (and) Aqua (water) (and) Laureth-3 (and) Hydroxyethylcellulose (and) Acetyl Dipeptide-1 Cetyl Ester; a mixture of Water (and) Erythritol (and) Homarine HCl; Centella asiatica; Biosaccharide Gum-1; Poly D-Glucosamine; 1-proline; 1-arginine; Retinol Microcaps; Hyaluronic acid; Dimethyl Isosorbide; Melaleuca alternafolia; a mixture of Cyclopentasiloxane (and) Dimethicone/Vinyl Dimethicone Crosspolymer; Phenoxyethanol SA; Benzylalcohol DHA; and Alpha Hydroxy acid.

Results

Each of the chronic wounds showed marked healing during use of the composition of the present invention, with several achieving nearly complete closure. Exemplary results are shown in the attached Figures, as described below.

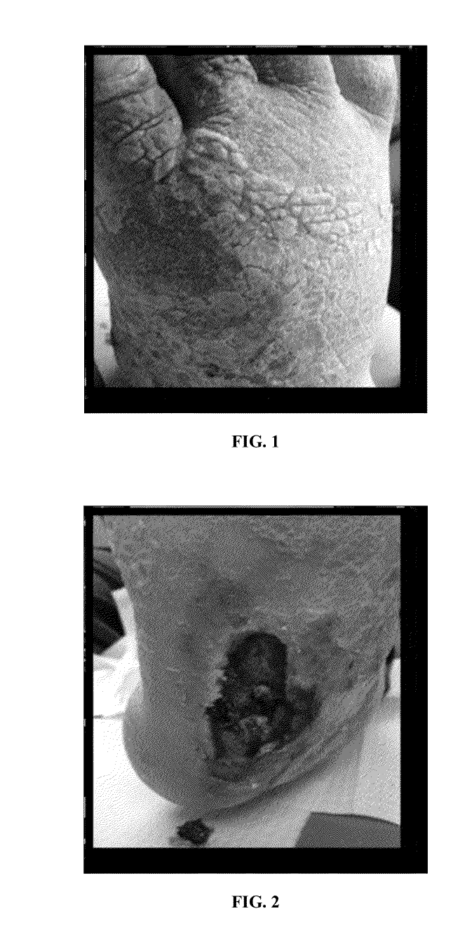

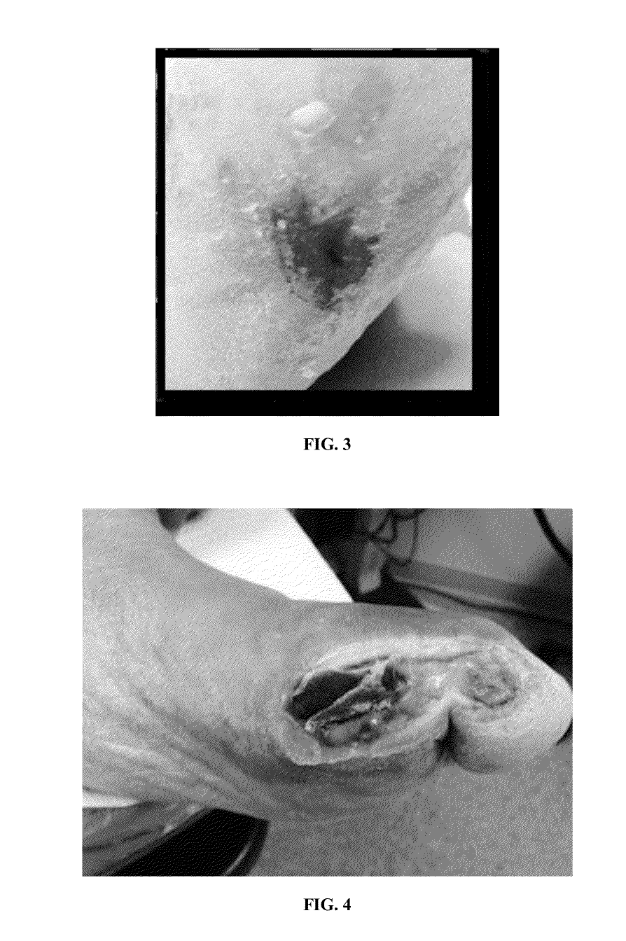

FIGS. 1, 2 and 3 show results in a 45 year old diabetic patient, post kidney transplant with a 3.5 year old infected wound. FIG. 1 shows the top of the subject's foot prior to application of a composition of the present invention. FIG. 2 shows the wound on the heel of the foot prior to application of a composition of the present invention. FIG. 3 shows the wound after 12 weeks of applying a composition of the present invention two times daily.

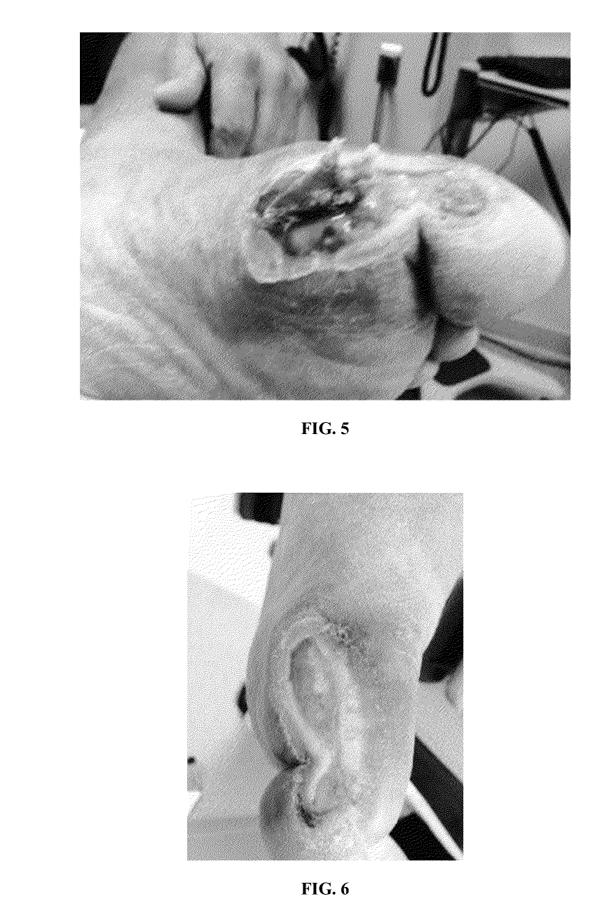

FIGS. 4, 5 and 6 show results in a 59 year old patient with a non-healing ulcer of 8 month duration having a thick eschar. Prior to application of a composition of the present invention the patient was on Bactrim and cultures were negative and X-rays and MRI negative for osteomyelitis. FIG. 4 shows the wound prior to application of a composition of the present invention. FIG. 5 shows the wound after 3 weeks of applying a composition of the present invention three times daily. FIG. 6 shows the wound after six weeks applying a composition of the present invention three times daily.

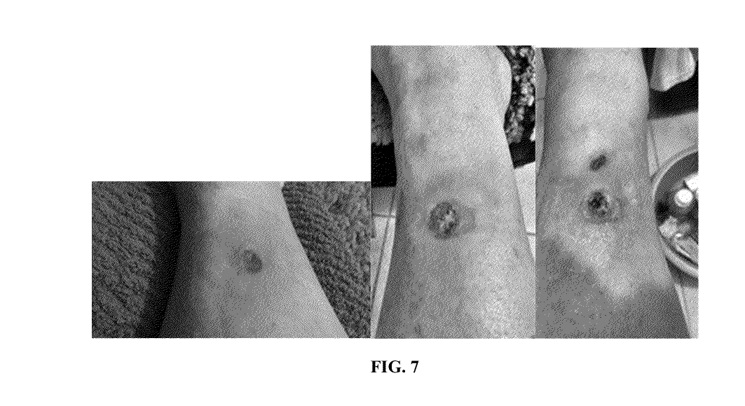

FIG. 7 shows results in a 75 year old patient with a non-healing three month old wound. The photo on the right shows the wound prior to application of a composition of the present invention. The middle photo shows the wound after 6 weeks of applying a composition of the present invention once daily. The photo on the left shows the wound after 12 weeks of applying a composition of the present invention once daily.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A method of making a stable composition comprising growth factors, said method comprising:
providing human umbilical cord blood plasma containing platelets, wherein said platelet-containing plasma is substantially free of whole blood cells;
lysing the platelets to extrude growth factors into the plasma;
encapsulating the growth factors contained within the plasma in a lipid bi-layer, wherein the resulting composition comprises encapsulated growth factors and remains stable and viable for at least 30 months.

2. The method of claim 1, wherein the encapsulating step comprises adding a propanediol and lecithin to the plasma to create one or more lipid bi-layers which form liposomes around the growth factors.

3. The method of claim 1, wherein the human umbilical cord blood plasma is obtained from at least two donors.

4. The method of claim 1, wherein the extruded growth factors are selected from the group consisting of platelet-derived growth factors and transforming growth factors.

5. The method of claim 1, wherein said lysing step is selected from the group consisting of traumatic lysing and protein induction.

6. The method of claim 5, wherein said traumatic lysing is selected from the group consisting of lysing by temperature shock, lysing by pH shock, lysing using high molecular weight fatty alcohols, and lysing by light emission.

7. The method of claim 6, wherein the traumatic lysing is lysing by temperature shock comprising:
freezing the platelet-containing plasma to between −20° C. and −30° C., and rapidly thawing the frozen plasma to between 32° C. and 39° C.

8. The method of claim 6, wherein the traumatic lysing is lysing by pH shock, comprising reducing the pH of the platelet-containing plasma to between 5.0 and 5.5.

9. The method of claim 6, wherein the traumatic lysing is lysing by light emission, comprising exposing the platelet-containing plasma to light emission in a visible range.

10. The method of claim 6, wherein the lysing step comprises protein induction by the addition of thromboglobulins.

11. The method of claim 1, further comprising the step of adding at least one matrikine peptide to the plasma, wherein said matrikine peptide is selected from the group consisting of gene receptor-stimulating matrikine peptides and matrix-derived peptides that regulate cell activity.

12. The method of claim 11, wherein the matrikine peptide is selected from the group consisting of Pal-GHK, Pal-GQPR.

13. The method of claim 11, wherein the matrikine peptide is in a solution comprising 0.25% to 4.0% matrikine peptides (v/v) and wherein the matrikine peptide solution comprises between 0.01% and 0.09% (v/v) of the platelet-containing plasma composition.

14. The composition produced by the method of claim 1.

15. The composition of claim 14 further comprising one or more additives selected from the group consisting of preservatives, emollients, delivery enhancers, penetrability enhancers, and moisturizers.

16. A method for using the composition of claim 15, wherein said method comprises applying the composition of claim 15 to a skin defect or wound on a subject's skin.

17. The method of claim 16, wherein the skin defect or wound is selected from the group consisting of a cosmetic skin defect, a traumatic skin defect, a chronic defect, a non-healing ulcer, an acute wound, a wound resulting from non-surgical/accidental trauma, a wound resulting from surgical trauma, a wound resulting from a chronic disease state, a wound resulting from topical irritation, trauma depleted fibroblasts, trauma depleted collagen, trauma depleted elastin, depleted adhesive plaque at the dermal/epidermal junction, damage caused by age-related skin deterioration, collagen alignment, scarring, scar formation, stretch marks, keloids, non-healing cratered ulcers, diabetic ulcers, cratered ulcers, diabetic neuropathies, hardened-cracked skin, hardened cracked heel tissue, and pressure ulcers, fine lines, wrinkles, and skin sagging.

18. A method of making a stable composition comprising growth factors, said method comprising:
providing human umbilical cord blood plasma containing platelets, wherein said platelet-containing plasma is substantially free of whole blood cells;
lysing the platelets to extrude growth factors into the plasma;
wherein said lysing step is selected from the group consisting of traumatic lysing and protein induction.

19. The method of claim 18, wherein said traumatic lysing is selected from the group consisting of lysing by temperature shock, lysing by pH shock, lysing using high molecular weight fatty alcohols, and lysing by light emission.

20. The method of claim 19, wherein the traumatic lysing is lysing by temperature shock comprising:
freezing the platelet-containing plasma to between −20° C. and −30° C.,
and rapidly thawing the frozen plasma to between 32° C. and 39° C.

21. The method of claim 19, wherein the traumatic lysing is lysing by pH shock, comprising reducing the pH of the platelet-containing plasma to between 5.0 and 5.5.

22. The method of claim 19, wherein the traumatic lysing is lysing by light emission, comprising exposing the platelet-containing plasma to light emission in a visible range.

23. The method of claim 18, wherein the lysing step comprises protein induction by the addition of thromboglobulins.
